(19) United States
(12) Patent Application Publication
(10) Pub. No.: US 2024/0008782 A1
(43) Pub. Date: Jan. 11, 2024

(54) SMART SYRINGE DEVICE, SYSTEM AND METHOD

(71) Applicant: THE REGENTS OF THE UNIVERSITY OF CALIFORNIA, Oakland, CA (US)

(72) Inventor: Ali Nsair, Westchester, CA (US)

(21) Appl. No.: 18/252,301

(22) PCT Filed: Nov. 9, 2021
(86) PCT No.: PCT/US21/58659

§ 371 Date: May 9, 2023

Related U.S. Application Data
(60) Provisional application No. 63/117,204, filed on Nov. 23, 2020.
(60) Provisional application No. 63/111,345, filed on Nov. 9, 2020.

(51) Int. Cl. A61B 5/157; A61B 5/145; A61B 5/1455; A61B 5/1468; A61B 5/153

(52) U.S. Cl. CPC A61B 5/157; A61B 5/14535; A61B 5/1455; A61B 5/1468; A61B 5/153; A61B 2562/0233; A61B 2560/0223; A61B 2560/0443; G01N 33/4905

(57) ABSTRACT

The present invention provides a smart syringe device that comprises an embedded impedance and optical sensors that allows for measuring activated clotting time (ACT) and Hemoglobin and Hematocrit (H&H) within the actual syringe without the need to disconnect the syringe from the patient.

### CROSS-REFERENCE TO RELATED APPLICATIONS

This application claims priority to U.S. Provisional Patent Application No. 63/111,345 filed Nov. 9, 2020, and U.S. Provisional Patent Application No. 63/117,204 filed Nov. 23, 2020, the contents of which are each incorporated by reference herein in their entirety.

### BACKGROUND OF THE INVENTION

Currently, conventional point of care blood tests are available for measuring activated clotting time (ACT) and Hemoglobin and Hematocrit (H&H) for medical procedures, surgeries, and disease diagnoses. Typically, these conventional systems and devices require several minutes for measurement and further require a blood-drawing syringe that must be disconnected from the patient to load a sample into a separate measurement apparatus. Such systems suffer from issues such as the introduction of dangerous pockets of air to the patient's blood streams, known as embolism and issues with blood coagulation which reduces the accuracy of such tests. Further, such systems require the use of heparin or other anticoagulants to prevent premature clotting which can impact the test results and lead to less accurate results.

Thus, there is a need in the art for a new blood testing system that can reduce life threating side effects such as embolism and improve the accuracy and speed of testing. The present invention meets this need.

### SUMMARY OF THE INVENTION

In one aspect the present invention provides a smart syringe device comprising: a syringe comprising a syringe chamber that is at least partially defined by a barrel wall; at least one electrical impedance sensor; at least one optical spectrometer; and a controller configured to provide an activated clotting time (ACT) measurement and Hemoglobin and a Hematocrit (H&H) measurement of blood within the syringe. In one embodiment, the at least one electrical impedance sensor comprises at least two electrodes and is positioned within the syringe chamber; and wherein the at least one optical spectrometer and the controller are positioned on an outer surface of the barrel wall. In one embodiment, the at least one optical spectrometer comprises a light source and a spectrometer. In one embodiment, the light source is a light emitting diode. In one embodiment, the light source is a laser diode. In one embodiment, the device further comprise an impedance calibration system having a top layer, a middle layer and a bottom layer; wherein the top layer comprises at least two reservoirs connected with at least one microfluidic channel positioned within the syringe chamber such that fluids drawn into the syringe chamber are flowable into the at least two reservoirs and the at least one microfluidic channel; wherein the bottom layer comprises at least two electrodes and wherein the top layer and the bottom layer are bonded together via the middle layer. In one embodiment, the at least one microfluidic channel comprises at least one chemical reagent configured to modulate a clotting reaction. In one embodiment, the middle layer comprises a pressure-sensitive adhesive. In one embodiment, the device further comprises an optical calibration system having a top layer, a middle layer, a bottom layer, a light source, and a spectrometer; wherein the top layer comprises at least one reservoirs positioned within the syringe chamber such that fluids drawn into the syringe chamber are flowable into the at least one reservoirs, wherein the bottom layer comprises a reflective optical surface and wherein the top layer and the bottom layer are bonded together via the middle layer. In one embodiment, the middle layer comprises a pressure-sensitive adhesive. In one embodiment, the controller is configured to analyze data from the at least one electrical impedance sensor.

In one aspect the present invention provides a smart syringe device comprising: a syringe having a barrel wall and a syringe chamber at least partially defined by the barrel wall; a disposable cartridge having a plurality of sections configured to receive a fluid from the syringe chamber, each of the plurality of sections comprising: at least one electrode and at least one reservoir fluidly connected to the syringe chamber through at least one microfluidic channel; and at least one printed circuit board positioned underneath each section comprising an impedance circuit, a light source and a spectrometer. In one embodiment, the light source is a light emitting diode. In one embodiment, the light source is a laser diode. In one embodiment, the impedance circuit comprises at least two spring-loaded pins that are connected to the at least one electrode. In one embodiment, the disposable cartridge is connected to a motor that is configured to rotate the cartridge such that a new section is positioned on top of the printed circuit for every new measurement. In one embodiment, the motor is configured to rotate the cartridge every 10-30 minutes. In one embodiment, the cartridge comprises four electrodes. In one embodiment, the at least one electrode comprises a clotting reagent.

In one aspect the present invention provides a method of providing an activated clotting time (ACT) measurement and a Hemoglobin and a Hematocrit (H&H) measurement comprising: providing a smart syringe device comprising a syringe having a syringe chamber at least partially defined by a barrel wall, at least one electrical impedance sensor having at least two electrodes positioned within the syringe chamber, and an electrical module positioned on an outer surface of the barrel wall comprising a light source, at least one photodetector and a controller; withdrawing blood from a patient using the syringe; drawing the blood into contact with the at least one electrical impedance sensor; generating a first signal from the at least one electrical impedance sensor; turning on the light source so that the light travels through the blood within the syringe chamber and enters the at least one photodetector; generating a second signal from the at least one photodetector; receiving the first and second signal by the controller; and calculating ACT based on the first signal and H&H based on the second signal.

In one aspect the present invention provides a method of providing an activated clotting time (ACT) measurement and a Hemoglobin and a Hematocrit (H&H) measurement comprising: providing a smart syringe device comprising a syringe; a cassette comprising at least one electrode, at least one reservoir and a printed circuit board, wherein the printed circuit board comprises an impedance circuit, a light source, and a spectrometer; a stopcock, wherein the stopcock selectively connects the syringe to a patient's vein or to the cassette; and a control unit; withdrawing blood from a patient using the syringe; changing the stopcock orientation such that the syringe is fluidly connected to the cassette; drawing the blood into contact with the at least one electrode and the at least one reservoir a first signal from at least one electrical impedance sensor; generating a first signal from the at least one electrode; generating a second signal by the spectrometer; receiving the first and second signal by the controller; and calculating ACT based on the first signal and H&H based on the second signal.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1B, depicts an exemplary smart syringe device of the present invention. FIG. 1 depicts a perspective view of an exemplary smart syringe device of the present invention. FIG. 1B depicts a cross-sectional view of an exemplary smart syringe device of the present invention.

FIG. 2 depicts an exemplary impedance calibration system of the present invention.

FIG. 3 depicts an exemplary optical calibration system of the present invention.

FIG. 4 depicts a perspective view of another exemplary smart syringe device of the present invention.

FIG. 5A through FIG. 5K depict perspective views of an exemplary smart syringe device of the present invention during operation.

FIG. 6 depicts a magnified view of an exemplary cassette of the present invention.

FIG. 7 depicts an exemplary smart syringe device of the present invention, featuring an electrical impedance system, and an optical absorption spectrometer.

FIG. 8 depicts how electrical impedance can rapidly and non-invasively measure viscosity of a fluid. Further, strongly linear relationship between blood viscosity and electrical impedance (r2=0.928; p<0.0001) measured from 10 unique subjects using a large-scale benchtop impedance system is shown.

FIG. 9 depicts strong linear relationship between concentration of hemoglobin using multi-frequency optical spectroscopy and conventional reagent-based laboratory instruments from blood contained in conventional IV bags (prediction coefficient 0.97; mean square error of 2.78 g/L) measured from 40 unique blood samples disposed in conventional blood bags that were not processed with any reagents.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of smart syringe systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

### Smart Syringe Device

The present invention relates to a smart syringe device that can be used for blood draws during any procedure and/or surgery. Embodiments of the smart syringe device features embedded impedance and optical sensors for measuring activated clotting time (ACT) and Hemoglobin and Hematocrit (H&H) within the actual syringe. The smart syringe device is configured to measure parameters including but not limited to ACT, H&H, etc. in just seconds while the syringe remains connected to the patient. The smart syringe device of the present invention allows immediate resulting of these tests for immediate follow-up and intervention if needed such as but not limited to adjusting the dose of infusing coagulants including but not limited to Heparin, direct thrombin inhibitor, etc. In one embodiment, the smart syringe device of the present invention allows for continuous measurements of parameters including but not limited to ACT, H&H, etc. The smart syringe device of the present invention improves upon conventional point-of-care systems for coagulation timing and blood analysis—these measurements conventionally requiring several minutes and a blood-drawing syringe that must be disconnected from the patient to load a sample into a separate measurement apparatus. Because the smart syringe device of the present invention makes measurements while attached to the patient, it also negates the possibility of air embolisms (air bubbles injected into the patient's venous or arterial system), a potentially life-threatening side effect that can occur when a conventional syringe is removed from the patient, carried over to a remote load point-of-care system, and then replaced. In one embodiment, the device of the present invention may be used on any patient on anticoagulation medicine including but not limited to heparin. In one embodiment, the device of the present invention may be used on any patient who is not in an anticoagulant infusion. In one embodiment, the device of the present invention is configured for use in any procedures, operating room, dialysis setting, etc. In one embodiment, the device may be used on patients in an inpatient setting. In one embodiment, the device may be used on the patients in an outpatient setting.

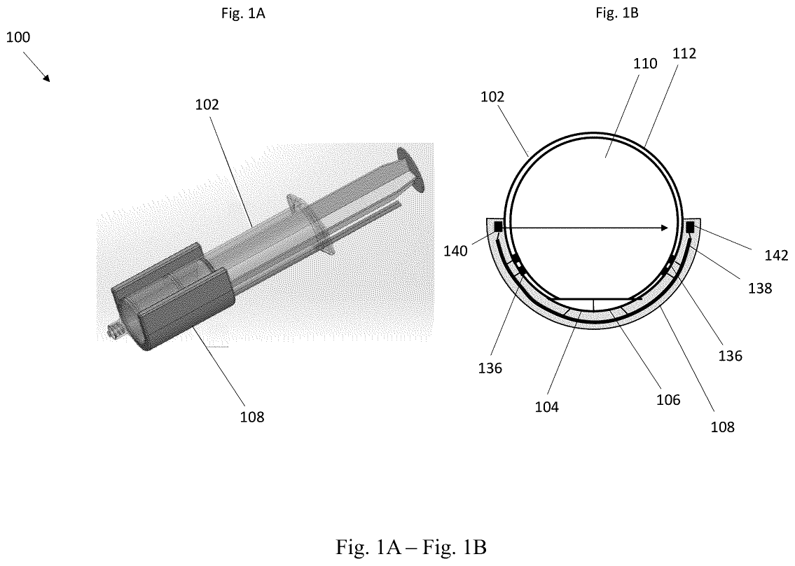

Referring now to FIG. 1A and FIG. 1B, an exemplary smart syringe device of the present invention is shown. Smart syringe device 100 comprises a syringe 102, at least one electrical impedance sensor, an impedance calibration system 104, an optical calibration system 106 and an electronic module 108 comprising a flex circuit 138, a light source 140, at least one photodetector 142, a microprocessor and a wireless transceiver.

Syringe 102 comprises a syringe chamber 110 that is at least partially defined by a barrel wall 112. In one embodiment, syringe 102 may be made from any material known to one skilled in the art including but not limited to polypropylene. In one embodiment, syringe 102 is made with transparent material to allow for optical measurements. In one embodiment, syringe 102 may have any diameter or length known to one skilled in the art. In one embodiment, syringe 102 may be disposable.

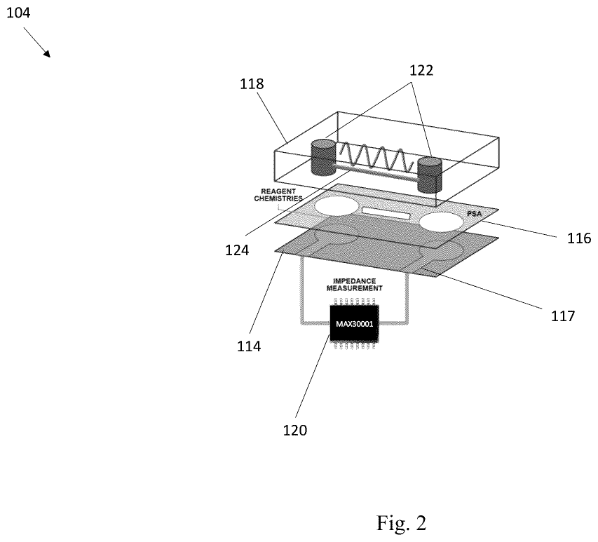

Referring now to FIG. 2, an exemplary impedance calibration system 104 of the present invention is shown. Impedance calibration system 104 is configured to make initial measurements of ACT (as impedance systematically increases as clotting progresses) and calibrate smart syringe device 100 to allow for an accurate, absolute measurement of ACT.

Impedance calibration system 104 comprises a bottom layer 114, a middle layer 116 and a top layer 118. Bottom layer 114 comprises at least two electrodes 117. In one embodiment, at least two electrodes 117 may be screen printed electrodes. In one embodiment, at least two electrodes 117 may be Ag/AgCl electrodes. In one embodiment, bottom layer 114 may be positioned in barrel wall 112, such that at least two electrodes 117 can be connected to an integrated circuit 120 positioned outside syringe chamber 110 in electronic module 108. Top layer 118 is positioned within syringe chamber 110 and comprises at least two reservoirs 122 such that fluids drawn into syringe chamber 110 are flowable into at least to reservoirs 122. At least two reservoirs 122 are connected via a microchannel 124. At least two reservoirs 122 are positioned on top of at least two electrodes 117. In one embodiment, at least two reservoirs 122 may have any cross-sectional shapes known to one skilled in the art including but not limited to circular, square, rectangular, etc. In one embodiment, at least two reservoirs 122 may have any volume known to one skilled in the art. In one embodiment, microchannel 124 may comprise at least one chemical reagent configured to modulate the clotting reaction. In one embodiment, microchannel 124 may have any length or width known to one skilled in the art.

In one embodiment, at least two reservoirs 122 and microchannel 124 may be made from any material known to one skilled in the art including but not limited to a polymer.

In one embodiment, bottom layer 114 and top layer 118 may be bonded together by middle layer 116. In one embodiment, middle layer 116 may be a pressure-sensitive adhesive. In one embodiment, middle layer 116 may comprise any other material known to one skilled in the art that is capable of bonding bottom layer 114 and top layer 118 together. In one embodiment, middle layer 116 may comprise at least one chemical reagent configured to modulate the clotting reaction.

In one embodiment, the at least one chemical reagent for measuring ACT may be any reagent known to one skilled in the art including but not limited to fibrinogen.

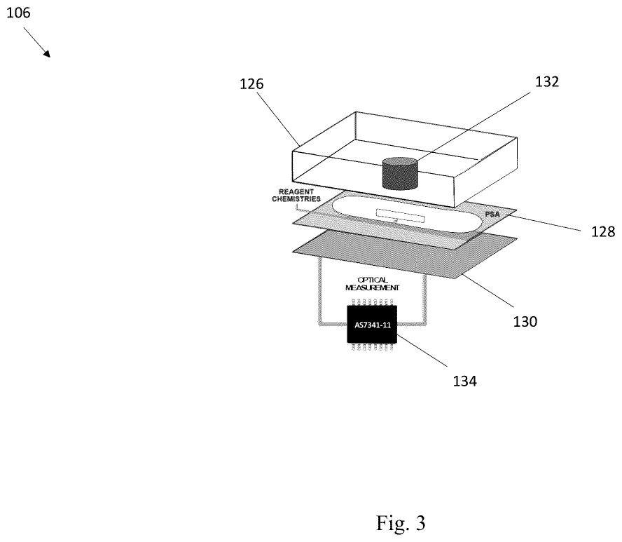

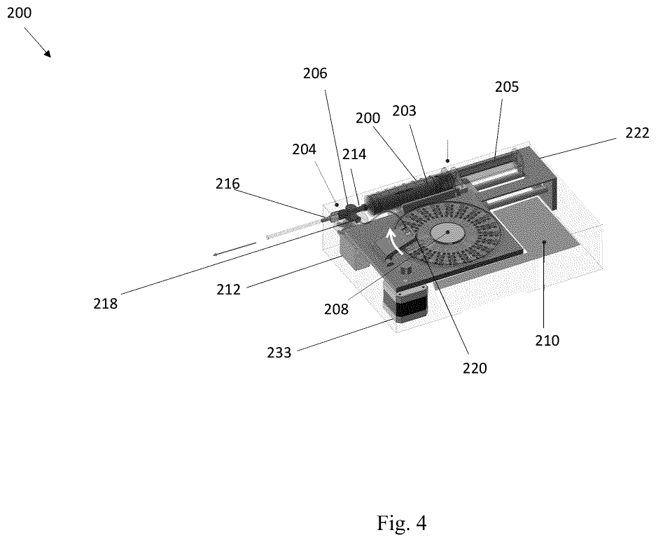

Referring now to FIG. 3, an exemplary optical calibration system 106 of the present invention is depicted. Optical calibration system 106 may be used to make initial measurements of H&H and calibrate smart syringe device 100. Optical calibration system 106 comprises a top layer 126, a middle layer 128, a bottom layer 130, a multi-frequency optical spectrometer 131 (FIG. 4) and a light source. Top layer 126 is positioned within syringe chamber 110 and comprises at least one reservoir 132 such that fluids drawn into syringe chamber 110 are flowable into at least one reservoir 132. In one embodiment, at least one reservoir 132 may have any cross-sectional shapes known to one skilled in the art including but not limited to circular, square, rectangular, etc. In one embodiment, at least one reservoir 132 may have any volume known to one skilled in the art. In one embodiment, at least one reservoir 132 may comprise at least one chemical reagent configured to modulate the clotting reaction. In one embodiment, at least one reservoir 132 may be made from any material known to one skilled in the art including but not limited to a polymer.

Bottom layer 130 comprises a reflective optical surface that is connected to an integrated circuit 134 positioned outside of syringe chamber 110 within electronic module 108.

In one embodiment, top layer 126 and bottom layer 130 may be bonded together by middle layer 128. In one embodiment, middle layer 128 may be a pressure-sensitive adhesive. In one embodiment, middle layer 128 may comprise any other material known to one skilled in the art that is capable of bonding top layer 126 and bottom layer 130 together. In one embodiment, middle layer 128 may comprise at least one chemical reagent configured to modulate the clotting reaction.

In one embodiment, the at least one chemical reagent for measuring H&H may be any reagent known to one skilled in the art including but not limited to sodium deoxycholate.

The multi-frequency optical spectrometer 131 is multiplexed and is configured to measure an optical transmission spectrum of the blood/reagent mixture. Both hemoglobin and hematocrit have distinct optical signatures, and thus levels of optical absorption yield initial values of H&H using conventional optical techniques (Tekkisin et al., 2012, Journal of Clinical Laboratory Analysis 26: 125-129). In one embodiment, the multi-frequency optical spectrometer 131 may be an 11-channel optical spectrometer (FIG. 4). In one embodiment, the multi-frequency optical spectrometer 131 may be a 7-channel optical spectrometer.

Optical calibration system 106 may use reflection-mode geometry to measure the H&H. In one embodiment, optical calibration system 106 may further comprise an optical window.

In one embodiment, the light source and the spectrometer may be positioned within electronic module 108. In one embodiment, the light source and spectrometer 131 may be positioned anywhere outside of syringe chamber 110.

At least one electrical impedance sensor is configured to measure the relative changes of ACT. In one embodiment, at least one electrical impedance sensor comprises at least two electrodes 136 configured to measure the electrical impedance of any fluid including but not limited to blood that is positioned within syringe chamber 110. In one embodiment, at least two electrodes 136 may be positioned within barrel wall 112 and extend towards an inner surface of syringe chamber 110, such that they are in contact with any fluid positioned inside syringe chamber 110 at one end and may be connected to an integrated circuit positioned outside of syringe chamber 110 within electronic module 108. In one embodiment, at least two electrodes 136 may be gold electrodes. In one embodiment, at least two electrodes 136 may be copper electrodes. In one embodiment, at least two electrodes 136 may be any other electrode known to one skilled in the art.

In one embodiment, at least one electrical impedance sensor may comprise four electrodes. The four electrodes may comprise two sense electrodes and two drive electrodes. In this configuration, during the measurement, the impedance circuit injects high-frequency, low-amperage current into drive electrodes and simultaneously measures a voltage drop induced by the injected current by connecting to the sense electrodes. In one embodiment, the impedance circuit may inject a high frequency ranging between 5-128 kHz. In one embodiment, the impedance circuit may inject a low amperage of 96 μAmps. In one embodiment, impedance circuit may be any circuit known to one skilled in the art including but not limited to MAX30001 (manufactured by Maxim Semiconductor). In one embodiment, at least one electrical impedance sensor may comprise any number of electrodes known to one skilled in the art.

As described above, electronic module 108 comprise a flex circuit 138, a light source 140, at least one photodetector 142, a microprocessor and a wireless transceiver. Electronic module 108 is positioned on the outer perimeter of barrel wall 112. In one embodiment, electronic module 108 may be removable. In one embodiment, electronic module 108 may be reusable between patients. In one embodiment, electronic module 108 may be sterilized by any method known to one skilled in the art. In one embodiment, electronic module 108 may be made from any material known to one skilled in the art including but not limited to plastic.

Flex circuit 138 is positioned within electronic module 108 and is configured to have a plurality of integrated circuits mounted thereto. In one embodiment, flex circuit 138 may be battery powered. In one embodiment, flex circuit 138 may be powered by a rechargeable source.

In one embodiment, light source 140 may be a light emitting diode (LED). In one embodiment light source 140 may be a laser diode. In one embodiment, the optical wavelengths may be visible. In one embodiment, the optical wavelengths may be infrared wavelengths. In one embodiment, the optical wavelengths may be near-infrared wavelengths.

At least one photodetector 142 is positioned on flex circuit 138 opposite to a position of light source 140 and is configured to measure an optical absorption spectrum of the fluid within syringe chamber 110 (as demonstrated by the arrow in FIG. 1B).

The microprocessor is configured to analyze any data from any of the integrated circuits 120, 134 mounted on flex circuit 138 using any algorithm known to one skilled in the art to yield continuous values of the measured parameters. Data from the microprocessor may be transmitted to an external display via a wireless transceiver. In one embodiment, the wireless transceiver may comprise a WiFi module or a Bluetooth module. In one embodiment, any other wireless module known to one skilled in the art may be used.

Referring now to FIG. 4, another exemplary smart syringe device 200 of the present invention is shown. Smart syringe device 200 comprises a syringe 202, a syringe pump 204, a stopcock 206, a cassette 208, a printed circuit board, a control unit 210 and a motor 233.

Syringe 202 comprises a syringe chamber 203 and a movable plunger 205. In one embodiment, syringe 202 may be any size, shape, or length known to one skilled in the art.

Syringe pump 204 comprises a housing 222 adapted to receive syringe 202, a motor and a controller unit. Syringe pump 204, driven by the motor, is configured to move plunger 205 forward or reverse and thereby allow loading or unloading fluid within syringe chamber 203 in a controlled manner using the control unit. In one embodiment, the motor may be any motor known to one skilled in the art including but not limited to a stepper motor. In one embodiment, the control unit is configured to receive input form a keypad, or other user input means, and various sensors, activate the motor and move plunger 203 in a forward or reverse direction. In one embodiment, the control unit may be connected to a display to allow a user to monitor the speed and direction of syringe 202 movement.

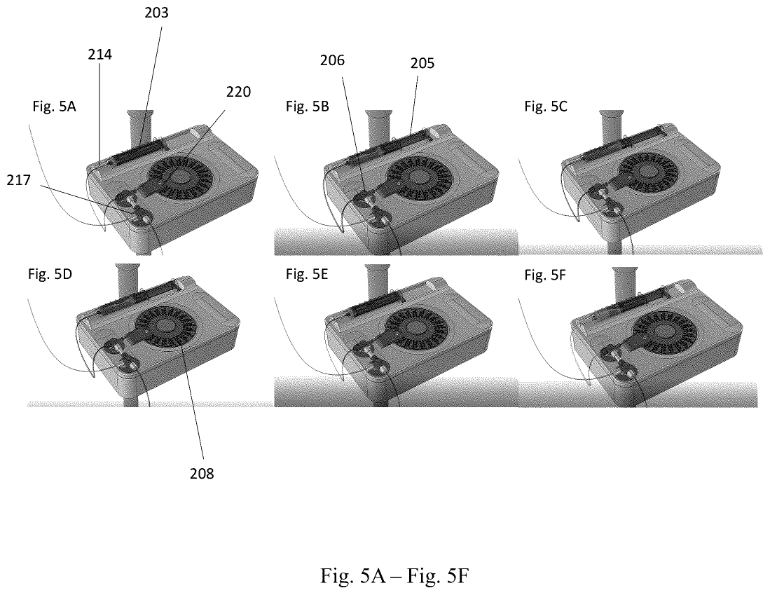

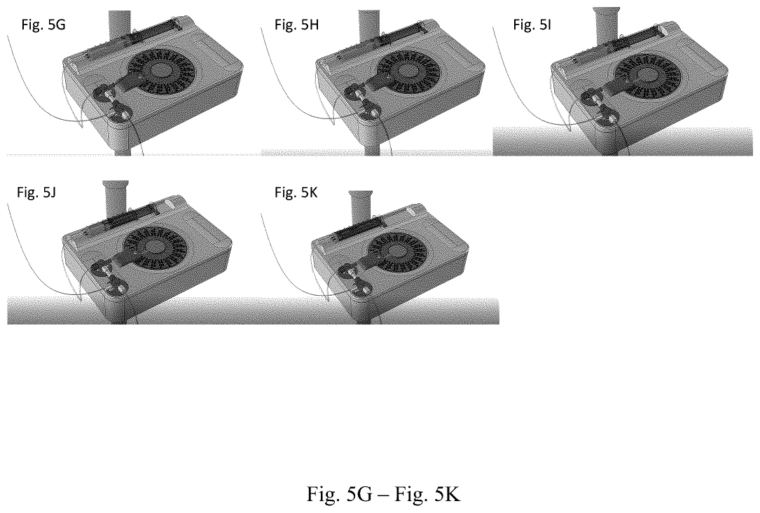

Stopcock 206 comprises a tubular body having a first tube section 214, a second tube section 216 and a third tube section 218. First tube section 214 is fluidly connected to syringe 202. Second tube section 216 is fluidly connectable to a patient's blood vessel. In one embodiment, the patient may be undergoing a medical procedure including but not limited to a cardiac catheterization. In one embodiment, second tube section 216 may be connected to a patient blood vessel via a second stopcock 217 (FIG. 5A through FIG. 5K). In one embodiment, second stopcock 217 may be connected to any external fluid source known to one skilled in the art.

Third tube section 218 is fluidly connected to cassette 208. Stopcock 206 is further connected to a motor 212 that is configured to open and close valves within stopcock 206 to switch the fluid pathway between a first position and a second position. In the first position, a fluid pathway between syringe 202 and the patient is open while the fluid pathway between syringe 202 and cassette 208 is closed. In the second position, a fluid pathway between syringe 202 and the patient is closed while the fluid pathway between syringe 202 and cassette 208 is open. In one embodiment, motor 212 may be any motor known to one skilled in the art including but not limited to a servo motor.

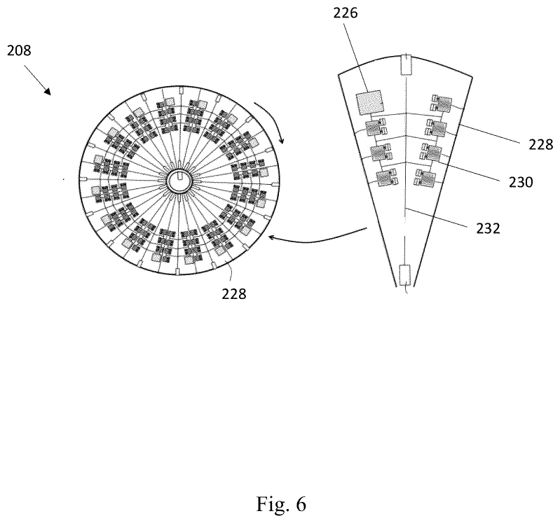

Referring now to FIG. 6, an exemplary cassette 208 is shown. In one embodiment, cassette 208 may comprise an inlet 220 and at least one section 228. In one embodiment, cassette 208 may be disposable.

Inlet 220 is fluidly connected to third tube section 218 and is configured to receive fluid from syringe 202. In one embodiment, cassette 208 may have 16 sections 228. In one embodiment, cassette 208 may have more than 16 sections 228. Each section 228 corresponds to a measurement panel and comprises at least one electrode 230, at least one reservoir 226 and at least one microfluidic channel 232. In one embodiment, each section 228 may comprise seven electrodes 230. In one embodiment, at least one electrode 230 may be plated with any material known to one skilled in the art. In one embodiment, at least one electrode 230 may be plated with gold. In one embodiment, at least one electrode 230 may be plated with copper. In one embodiment, at least one electrode 230 may be coated with any chemical reagents known to one skilled in the art. Chemical reagents may be able to initiate a chemistry on at least one electrode 230 and better measure certain parameters.

At least one electrode 230 and at least one reservoir 226 are connected to inlet 220 through at least one microfluidic channel 232. In one embodiment, at least one electrode 230 and at least one reservoir 226 may be configured to measure ACT and H&H. In one embodiment, at least one electrode 230 and at least one reservoir 226 may be able to measure other fluid parameters including but not limited to pH, glucose, lactate, potassium levels, etc.

The printed circuit board is positioned underneath at least one section 228 and comprises an impedance circuit, a broadband light source, and a spectrometer. In one embodiment, a unique impedance circuit corresponds to each one of the at least one electrode 230. In one embodiment, the impedance circuit comprises at least two spring-loaded pins that is connected to each sense and drive component of at least one electrode 230 and functions as described elsewhere herein.

In one embodiment, at least one section 228 may comprise four electrodes 230 with conductive copper traces that can be filled with a small volume of blood. The copper traces are connected to an impedance circuit. In one embodiment, the four electrodes may comprise a clotting reagent including but not limited to sodium citrate to expedite the clotting process. In one embodiment, the four electrodes comprise two sense electrodes and two drive electrodes. In this configuration, during the measurement, the impedance circuit injects high-frequency, low-amperage current into drive electrodes and simultaneously measures a voltage drop induced by the injected current by connecting to the sense electrodes. As blood begins to gel, its impedance (i.e., resistance) gradually decreases. The impedance circuit measures and digitizes the voltage drop to generate a time-dependent impedance waveform, which is then stored and analyzed with an algorithm to estimate the blood's ACT. In one embodiment, the four electrodes may be augmented with an additional measurement called ‘bioreactance’ to detect phase changes of electrical current flowing through the blood sample; this yields a time-dependent waveform that also indicates ACT and other parameters within the blood (e.g., water content). The combination of impedance and bioreactance further refines the accuracy of measurements of ACT and other parameters.

In one embodiment, the at least one electrode 230 may be free of any clotting reagent. Here the impedance measurements can yield the blood's viscosity, which is related to ACT by a pre-determined linear regression formula. This approach has an important advantage that it can be done very rapidly (e.g., in a matter of seconds). In still other embodiments, a ‘hybrid’ measurement is made where the reagent-based ACT measurement is performed with one electrode to yield an initial ‘ACT calibration’. The system then makes the above-described measurements of viscosity with a reagent-free electrode and combines these with the ACT calibration to yield accurate (and rapid) follow-on measurements of ACT.

In one embodiment, a single broadband light source and a spectrometer corresponds to at least one reservoir 226. During a measurement, the at least one reservoir may be filled with blood. For optical absorption measurements, the at least one reservoir is filled with blood, and is irradiated by light from the broadband light source. Radiation reflects off the blood sample and into the spectrometer, which then measures an optical absorption spectrum that is digitized, stored, and analyzed to determine H&H.

In one embodiment, the light source may be a light emitting diode (LED). In one embodiment the light source may be a laser diode. In one embodiment, the optical wavelengths may be visible. In one embodiment, the optical wavelengths may be infrared wavelengths. In one embodiment, the optical wavelengths may be near-infrared wavelengths.

The spectrometer is multiplexed and is configured to measures an optical transmission spectrum of the blood/reagent mixture positioned within each section 228. Both hemoglobin and hematocrit have distinct optical signatures, and thus levels of optical absorption yield initial values of H&H using conventional optical techniques (Tekkisin et al., 2012, Journal of Clinical Laboratory Analysis 26: 125-129). In one embodiment, the spectrometer may be an 11-channel optical spectrometer. In one embodiment, the spectrometer may be a 7-channel optical spectrometer. The spectrometer may use reflection-mode geometry to measure the H&H. In one embodiment, an optical window may also be used for optical measurements.

Motor 233 is configured to rotate cassette 208 to expose a new section 228 such that the new section 228 receive a sample of the fluid from syringe chamber 203 and is exposed to the light source and is connected to the impedance circuit. Once a set of measurements on one section is done, motor 233 is activated and rotates cassette 208 such that a new section 228 is disposed, allowing new measurements to be made. In one embodiment, motor 233 rotates every minutes to allow new measurements on a new section 228.

Control unit 210 is configured to analyze any data from impedance circuit or the spectrometer using any algorithm known to one skilled in the art to yield continuous values of the measured parameters. Data from control unit 210 may be transmitted to an external display via a wireless transceiver. In one embodiment, the wireless transceiver may comprise a WiFi module or a Bluetooth module. In one embodiment, any other wireless module known to one skilled in the art may be used. In one embodiment, control unit 210 may be configured to store any data from impedance circuit or the spectrometer.

In one embodiment, device 200 is configured to allow simultaneous measurement of multiple fluid parameters. In one embodiment, device 200 is configured to allow continuous measurement of multiple fluid parameters.

### Method of Use

The present invention relates to methods of providing an activated clotting time (ACT) measurement and a Hemoglobin and a Hematocrit (H&H) measurement from a blood sample while the blood is in the syringe chamber without removing the syringe from a patient. In one embodiment, the method of present invention

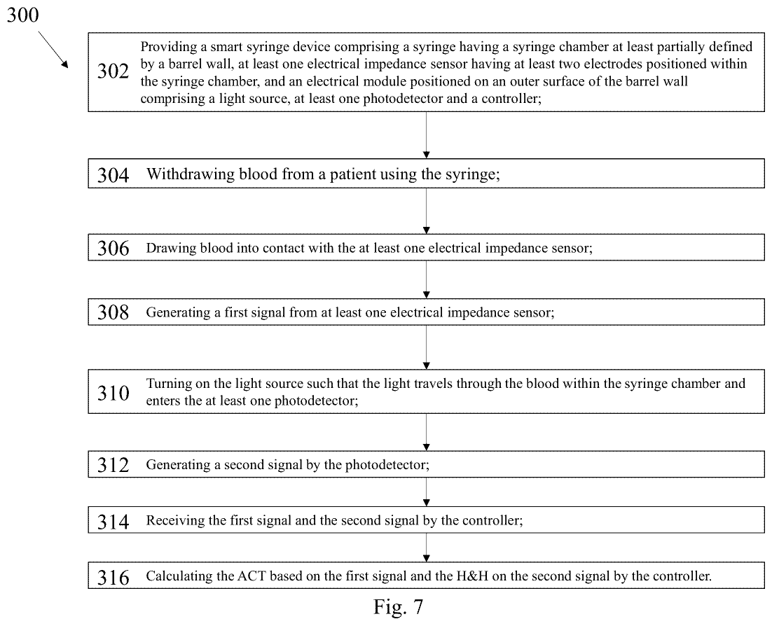

Referring now to FIG. 7, an exemplary method of providing an ACT measurement and a H&H measurement is depicted. Method 300 begins with step 302 wherein a smart syringe device is provided, the smart syringe device comprising a syringe having a syringe chamber at least partially defined by a barrel wall, at least one electrical impedance sensor having at least two electrodes positioned within the syringe chamber, and an electrical module positioned on an outer surface of the barrel wall comprising a light source, at least one photodetector and a controller. In step 304, blood from a patient is withdrawn using the syringe. In step 306, blood is drawn into contact with the at least one electrical impedance sensor. In step 308, a first signal is generated from at least one electrical impedance sensor. In step 310, the light source is turned on such that the light travels through the blood within the syringe chamber and enters the at least one photodetector. In step 312, a second signal is generated by the photodetector. In step 314, the controller receives the first signal and the second signal. In step 316, the controller calculates ACT based on the first signal and H&H based on the second signal.

In one embodiment, the smart syringe device further comprises an impedance calibration system as described elsewhere herein. In one embodiment, the smart syringe device further comprises an optical calibration system as described elsewhere herein.

In one embodiment, the method may further comprise a step of determining an ACT value of the blood based on a previously measured initial value of ACT. In one embodiment, the method includes a step of determining a H&H value based on a previously measured initial value of H&H.

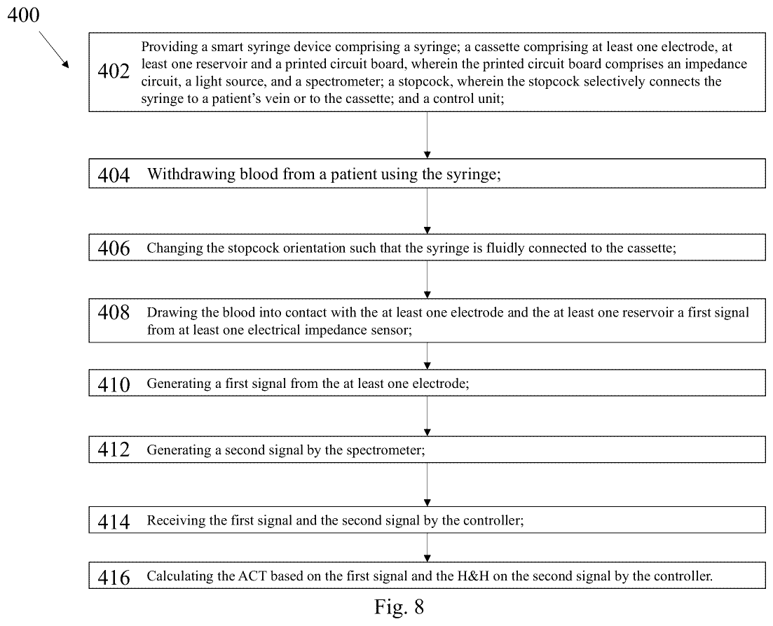

Referring now to FIG. 8, another exemplary method of providing an ACT measurement and a H&H measurement is depicted. Method 400 begins with step 402, wherein a smart syringe device is provided, the smart syringe device comprising a syringe; a cassette comprising at least one electrode, at least one reservoir and a printed circuit board, wherein the printed circuit board comprises an impedance circuit, a light source, and a spectrometer; a stopcock, wherein the stopcock selectively connects the syringe to a patient's vein or to the cassette; and a control unit. In step 404, blood from a patient is withdrawn using the syringe. In step 404, changing the stopcock orientation such that the syringe is fluidly connected to the cassette. In step 406, blood is drawn into contact with the at least one electrode and the at least one reservoir. In step 408, a first signal is generated from the at least one electrode. In step 410, the light source is turned on such that the light travels through the fluid within the at least one reservoir and enters the spectrometer. In step 412, a second signal is generated by the spectrometer. In step 414, the control unit receives the first signal and the second signal. In step 416, the control unit calculates ACT based on the first signal and H&H based on the second signal. In one embodiment, the control unit may be able to measure other fluid parameters including but not limited to pH, glucose, lactate, potassium levels, etc.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out exemplary embodiments of the present invention, and are not to be construed as limiting in any way the remainder of the disclosure.

### Example 1

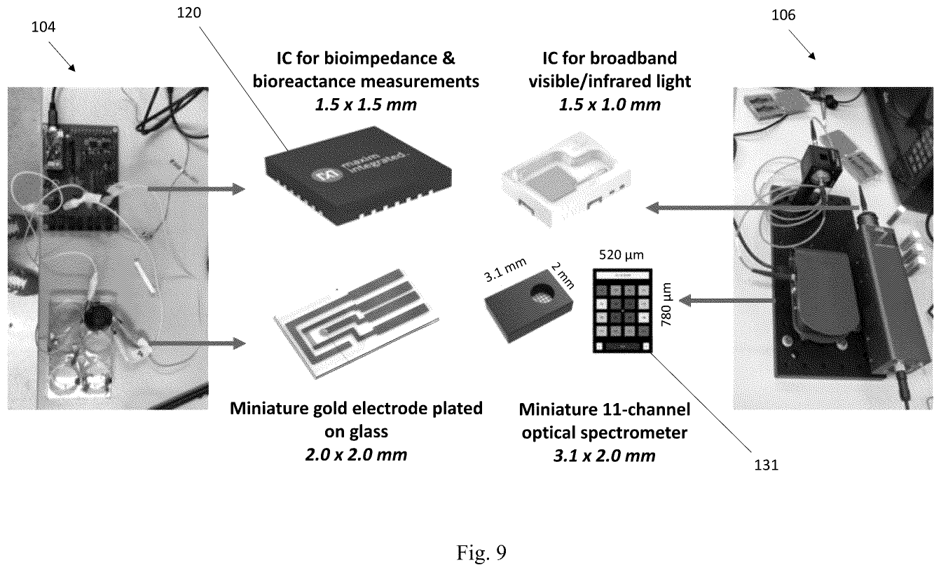

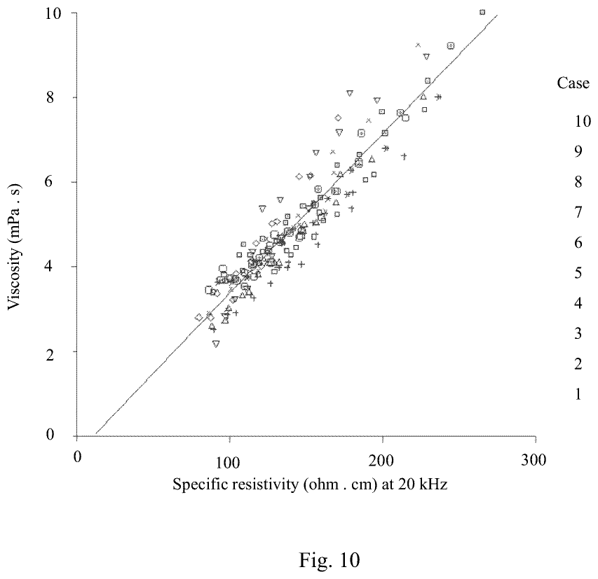

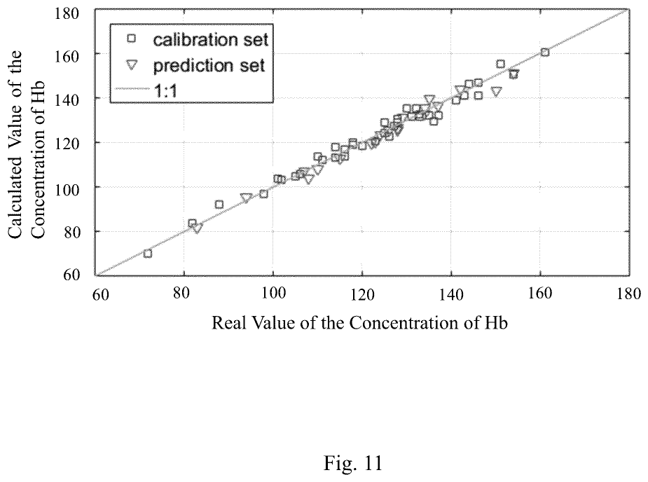

Referring now to FIG. 9 an exemplary smart syringe device of the present invention is shown. The electrical impedance system features four copper electrodes deposited on a glass plate featuring a reservoir that can be filled with a small volume of blood. The electrodes attach through cables to a circuit board that features an impedance circuit. Two distal ‘drive’ electrodes in the glass plate inject high-frequency, low-amperage current generated by the impedance circuit; two inner ‘sense’ electrodes measure a voltage drop induced by the injected current. As blood within the reservoir clots, its impedance (i.e., resistance) decreases as it gels. The impedance circuit measures and digitizes the voltage drop to generate a time-dependent impedance waveform, which is then stored and analyzed. An off-line algorithm processes the impedance waveforms to estimate the activated clotting time (ACT) of blood with clotting within the reservoir.

### Example 2

### Electrical Impedance and ACT:

Clinical studies performed internally, along with those described in the medical literature, indicate how electrical impedance can rapidly and non-invasively measure viscosity of a fluid, and most notably human blood (Pop et al., 2004, Eur Surg Res; 36:259-265; Berney et al., 2008, Analog Dialogue 42-08). See FIG. 10, which shows the strongly linear relationship between blood viscosity and electrical impedance (r2=0.928; p<0.0001) measured from 10 unique subjects using a large-scale benchtop impedance system.

Relative changes in blood viscosity, in turn, indicate corresponding changes in Heparin levels and ACT (Hiosugi et al., 2001, 104:371-374; Ranucci et al., 2014, Physiol Rep, 2 (7): e12065). This indicates that electrical impedance represents a potential measurement for determining relative changes in ACT in blood without using reagents. However, electrical impedance systems that operate in this point-of-care capacity—i.e. directly on the patient—have proven elusive, mostly because historically such systems are large, cumbersome, and relegated to remote, boxy instruments. In clinical practice impedance measurement of blood properties, while effective, have been largely surpassed by benchtop methodologies that assess the ACT by more conventional methodologies, such as mechanical or photo-optical detection of thrombin-induced clot formation (Ranucci et al., 2014, Physiol Rep, 2 (7): e12065; Dirkman et al., 2019, BMC Anesthesiol. Sep. 6; 19(1):174; Ojito et al., 2012, JECT 44:15-20).

Making point-of-care measurements possible is the small integrated circuit (e.g., the MAX30001), roughly 1.5×1.5 mm, that performs an electrical impedance measurement suitable to generate data like that shown in FIG. 10. During a measurement, the circuit injects a high-frequency (5-128 kHz) low-amperage (96 μAmps) current into 2 distal, blood-contacting gold electrodes plated on the inner surface of the syringe; these are the ‘drive’ electrodes. Simultaneously the circuit connects to interior electrodes proximal to the drive electrodes that contact blood within the syringe and measure a corresponding voltage drop; these are the ‘sense’ electrodes. During the coagulation process blood changes from a liquid to a solid gel phase, resulting in corresponding changes in blood viscosity and as indicated by FIG. 10, electrical impedance. Algorithms operating on a microprocessor receive impedance values from the circuit and then convert these into relative changes in ACT as described above.

### Optical Spectroscopy and H&H:

Visible and near-infrared transmission optical spectroscopy are well established for determining absolute hemoglobin and hematocrit concentration from blood samples processed with clotting reagents (Zhang et al., 2018, Scientific reports 8(1): 1-9). Recent work indicates that relative changes in these parameters can be determined using comparable optical techniques and no reagents (Whitehead et al., 2019, Annals of the New York Academy of Sciences 1450.1: 147). See FIG. 11, which shows the strongly linear relationship between concentration of hemoglobin using multi-frequency optical spectroscopy and conventional reagent-based laboratory instruments from blood contained in conventional IV bags (prediction coefficient 0.97; mean square error of 2.78 g/L) measured from 40 unique blood samples disposed in conventional blood bags that were not processed with any reagents.

However, as with electrical impedance, conventional spectroscopic systems used for such tests are typically large and cumbersome, thereby relegating them to benchtop systems.

The multi-frequency spectrometer (e.g., the AS7341-11) in the smart syringe makes optical measurements for H&H similar to those shown in FIG. 11; they are made directly on the patient to determine relative H&H values. This small integrated system, measuring roughly 3×2 mm in size, features 8 optical channels distributed over the visible range, an additional near-infrared channel, and 6 parallel analog-to-digital converters for on-board signal processing. When coupled with the smart syringe's on-board processor, this gives the system the same spectroscopic capabilities for measuring optical signals as the benchtop spectrometers, described above.

Similarly, for optical measurements, the multi-frequency optical spectrometer is also multiplexed and measures an optical transmission spectrum of the blood/reagent mixture. Both hemoglobin and hematocrit have distinct optical signatures, and thus levels of optical absorption yield initial values of H&H using conventional optical techniques (Zhang et al., 2018, Scientific reports 8(1): 1-9).

1-20. (canceled)

21. A smart syringe device comprising:
a syringe comprising a syringe chamber that is at least partially defined by a barrel wall;
at least one electrical impedance sensor;
at least one optical spectrometer; and
a controller configured to provide an activated clotting time (ACT) measurement and Hemoglobin and a Hematocrit (H&H) measurement of blood within the syringe.

22. The smart syringe device of claim 21, wherein the at least one electrical impedance sensor comprises at least two electrodes and is positioned within the syringe chamber; and wherein the at least one optical spectrometer and the controller are positioned on an outer surface of the barrel wall.

23. The smart syringe device of claim 22, wherein the at least one optical spectrometer comprises a light source and a spectrometer.

24. The smart syringe device of claim 23, wherein the light source is a light emitting diode.

25. The smart syringe device of claim 23, wherein the light source is a laser diode.

26. The smart syringe device of claim 21, further comprising an impedance calibration system having a top layer, a middle layer and a bottom layer; wherein the top layer comprises at least two reservoirs connected with at least one microfluidic channel positioned within the syringe chamber such that fluids drawn into the syringe chamber are flowable into the at least two reservoirs and the at least one microfluidic channel; wherein the bottom layer comprises at least two electrodes and wherein the top layer and the bottom layer are bonded together via the middle layer.

27. The smart syringe device of claim 26, wherein the at least one microfluidic channel comprises at least one chemical reagent configured to modulate a clotting reaction.

28. The smart syringe device of claim 26, wherein the middle layer comprises a pressure-sensitive adhesive.

29. The smart syringe device of claim 22, further comprising an optical calibration system having a top layer, a middle layer, a bottom layer, a light source, and a spectrometer; wherein the top layer comprises at least one reservoirs positioned within the syringe chamber such that fluids drawn into the syringe chamber are flowable into the at least one reservoirs, wherein the bottom layer comprises a reflective optical surface and wherein the top layer and the bottom layer are bonded together via the middle layer.

30. The smart syringe device of claim 29, wherein the middle layer comprises a pressure-sensitive adhesive.

31. The smart syringe device of claim 22, wherein the controller is configured to analyze data from the at least one electrical impedance sensor.

32. A smart syringe device comprising:
a syringe having a barrel wall and a syringe chamber at least partially defined by the barrel wall;
a disposable cartridge having a plurality of sections configured to receive a fluid from the syringe chamber, each of the plurality of sections comprising: at least one electrode and at least one reservoir fluidly connected to the syringe chamber through at least one microfluidic channel; and
at least one printed circuit board positioned underneath each section comprising an impedance circuit, a light source and a spectrometer.

33. The smart syringe device of claim 32, wherein the light source is a light emitting diode.

34. The smart syringe device of claim 32, wherein the light source is a laser diode.

35. The smart syringe device of claim 32, wherein the impedance circuit comprises at least two spring-loaded pins that are connected to the at least one electrode.

36. The smart syringe device of claim 32, wherein the disposable cartridge is connected to a motor that is configured to rotate the cartridge such that a new section is positioned on top of the printed circuit for every new measurement.

37. The smart syringe device of claim 36, wherein the motor is configured to rotate the cartridge every 10-30 minutes.

38. The smart syringe device of claim 32, wherein the cartridge comprises four electrodes.

39. The smart syringe device of claim 32, wherein the at least one electrode comprises a clotting reagent.

40. A method of providing an activated clotting time (ACT) measurement and a Hemoglobin and a Hematocrit (H&H) measurement comprising:
providing a smart syringe device comprising a syringe having a syringe chamber at least partially defined by a barrel wall, at least one electrical impedance sensor having at least two electrodes positioned within the syringe chamber, and an electrical module positioned on an outer surface of the barrel wall comprising a light source, at least one photodetector and a controller;
withdrawing blood from a patient using the syringe;
drawing the blood into contact with the at least one electrical impedance sensor;
generating a first signal from the at least one electrical impedance sensor;
turning on the light source so that the light travels through the blood within the syringe chamber and enters the at least one photodetector;
generating a second signal from the at least one photodetector;
receiving the first and second signal by the controller; and
calculating ACT based on the first signal and H&H based on the second signal.

41. A method of providing an activated clotting time (ACT) measurement and a Hemoglobin and a Hematocrit (H&H) measurement comprising:
providing a smart syringe device comprising a syringe; a cassette comprising at least one electrode, at least one reservoir and a printed circuit board, wherein the printed circuit board comprises an impedance circuit, a light source, and a spectrometer; a stopcock, wherein the stopcock selectively connects the syringe to a patient's vein or to the cassette; and a control unit;
withdrawing blood from a patient using the syringe;
changing the stopcock orientation such that the syringe is fluidly connected to the cassette;
drawing the blood into contact with the at least one electrode and the at least one reservoir a first signal from at least one electrical impedance sensor;
generating a first signal from the at least one electrode;
generating a second signal by the spectrometer;
receiving the first and second signal by the controller; and
calculating ACT based on the first signal and H&H based on the second signal.
